SEQUENCE LISTING (1 sequence)

SEQ ID NO: 1 - DNA, 759 bp, Artificial Sequence
  ISF35 nucleic acid sequence
atgatcgaaacatacaaccaaacttctccccgatctgcggccactggactgcccatcagcatgaaaatttttatgtatttacttactgtttttcttatcacccagatgattgggtcagcactttttgctgtgtatcttcatagaaggctggacaagatagaagatgaaaggaatcttcatgaagattttgtattcatgaaaacgatacagagatgcaacacaggagaaagatccttatccttactgaactgtgaggagattaaaagccagtttgaaggctttgtgaaggatataatgttaaacaaagaggagacgaagaaagatgaggatcctcaaattgcagcacacgttgtaagcgaagccaacagtaatgcagcatccgttctacagtgggccaagaaaggatattataccatgaaaagcaacttggtaaccctggaaaatgggaaacagctgacggttaaaagacaaggactctattatatctatgctcaagtcaccttctgctctaatcgggaggcttcgagtcaagccccattcatcgtcggcctctggctgaagcccagcagtggatctgagagaatcttactcaaggcggcaaatacccacagttcctcccagctttgcgagcagcagtctattcacttgggcggagtgtttgaattacaaccaggtgcttcggtgtttgtcaatgtgactgatccaagccaagtgagccatggcactggcttcacgtcctttggcttactcaaactctga

(19) United States
(12) Patent Application Publication
(10) Pub. No.: US 2018/0244750 A1
(43) Pub. Date: Aug. 30, 2018

(54) METHODS FOR TREATMENT OF CANCER

(71) Applicant: Memgen, LLC, Houston, TX (US)

(72) Inventor: Mark J. Cantwell, San Diego, CA (US)

(73) Assignee: Memgen, LLC, Houston, TX (US)

(21) Appl. No.: 15/773,090

(22) PCT Filed: Nov. 2, 2016
(86) PCT No.: PCT/US2016/060114

§ 371 Date: May 2, 2018

Related U.S. Application Data
(60) Provisional application No. 62/249,935, filed on Nov. 2, 2015.

(51) Int. Cl. C07K 14/705; A61P 35/00; C12N 7/00; G01N 33/574; A61K 39/395; A61K 38/17

(52) U.S. Cl. CPC C07K 14/70575; A61P 35/00; C12N 7/00; G01N 33/57492; A61K 48/00; A61K 38/177; C12N 2710/10021; C12N 2710/10071; C12N 2710/10043; A61K 39/39558

(57) ABSTRACT

Provided herein are methods of sensitizing cancer cells to treatment with inhibitors of the PD-1 pathway. Such methods comprise treatment of subject with a checkpoint inhibitor refractory cancer with an expression vector encoding a chimeric CD154.

The present application is filed in the US National Stage under 35 U.S.C. 371 from co-pending International Application No. PCT/US16/60114, filed on Nov. 2, 2016, which claims the benefit under 35 U.S.C. § 119(e) of prior-filed co-pending provisional application No. 62/249,935, filed Nov. 2, 2015.

### BACKGROUND

### Field of Invention

Provided herein are methods and compositions for the treatment of cancer.

### Description of Related Art

An immune reaction typically begins with a T lymphocyte (T cell) that has on its surface a T cell receptor (TCR) that binds to an antigen-derived peptide associated with a class II major histocompatibility complex (MHC) molecule. The T cell also expresses on its surface various polypeptides, which are referred to as ligands. When the T cell receptor binds to a MHC-associated antigen, for example an antigen derived from a malignant cell, it becomes activated and expresses a ligand on its surface. The ligand is only present on the cell surface for a short time, and once it has been removed from the surface of the cell, the T cell's ability to bind a receptor-bearing cell is lost.

One such ligand is called CD154, a member of a larger family of ligands, collectively referred to as the TNF superfamily (Cross et al, Cytokines Mol Ther, 1:75-105, 1995 and Locksley et al, Cell, 104:487-501, 2001). TNF superfamily members share a conserved secondary structure comprising four domains: domain 1, the intracellular domain; domain II, which spans the cell membrane and is known as the transmembrane domain; domain III, which consists of the extracellular amino acids closest to the cell membrane; and domain IV, the distal extracellular domain (Kipps et al., WO98/2606 I published Jun. 18, 1998). Typically, at least a part of domain IV can be cleaved from the parent molecule. The cleaved fragment often exhibits the same biological activity of the intact ligand and is conventionally referred to as a “soluble form” of the TNF family member.

The interactions between CD154 (also known as CD40 ligand) and its cognate receptor, CD40, are critical for immune recognition (Banchereau J. et al., Annu. Rev. Immunol. 12:881-922, 1994; Laman J. D. et al., Crit. Rev. Immunol., 16:59-108, 1996). CDI54 is transiently expressed on CD4+ T cells following T cell receptor engagement by antigen-presenting cells through MHC class II molecules (Roy M. et al., J. Immunol., 151:2497-2510, 1993; Hepmann P. et al., Eur. J. Immunol., 23:961-964, 1993; Castle B. E. et al., J. Immunol., 151: 1777-1788, 1993; Cantwell M. et al., Nat. Med., 3:984-989, 1997). This, in turn, can cause activation ofCD40-expressing antigen presenting cells (APCs), including B cells, dendritic cells, monocytes, and macrophages (Ranheim E. A. et al., J. Exp. Med., 177:925-935, 1993; Ranheim E. A. et al., Cell. Immunol., 161:226-235, 1995). Such CD40 activated cells can set off a cascade of immune activating events that lead to a specific and effective immune response against foreign antigens, such as viruses or tumors. The importance of interactions between CD40 and CD154 is underscored by the finding that individuals who have inherited defects in the ligand for CD40 have profound immune deficiency (Korthauer J. et al., Nature, 361:539-541, 1993, Aruffo A. et al., Cell., 72:291-300, 1993). Such patients have an immune deficiency syndrome associated with impaired germinal center formation, defective isotype switching, and marked susceptibility to various bacterial and viral pathogens.

The program cell death protein 1 (PD-1) pathway is recognized as an important player in immune regulation and the maintenance of peripheral tolerance. PD-1 is moderately expressed on naive T, Band NKT cells and up-regulated by T/B cell receptor signaling on lymphocytes, monocytes and myeloid cells.

Two known ligands for PD-1, program cell death protein 1 ligand 1 (PD-L1; B7-H1) and program cell death protein 1 ligand 2(PD-L2; B7-DC), are expressed in human cancers arising in various tissues. In large sample sets of, for example, ovarian, renal, colorectal, pancreatic, liver cancers and melanoma, it was shown that PD-L1 expression correlated with poor prognosis and reduced overall survival irrespective of subsequent treatment. Similarly, PD-1 expression on tumor infiltrating lymphocytes was found to mark dysfunctional T cells in breast cancer and melanoma and to correlate with poor prognosis in renal cancer. Thus, it has been proposed that PD-L1 expressing tumor cells interact with PD-1 expressing T cells to attenuate T cell activation and evasion of immune surveillance, thereby contributing to an impaired immune response against the tumor. Several monoclonal antibodies that inhibit the interaction between PD-1 and one or both of its ligands PD-L1 and PD-L2 are in clinical development for treating cancer. It has been proposed that the efficacy of such antibodies might be enhanced if administered in combination with other approved or experimental cancer therapies, e.g., radiation, surgery, chemotherapeutic agents, targeted therapies, agents that inhibit other signaling pathways that are dysregulated in tumors, and other immune enhancing agents.

### SUMMARY OF INVENTION

Provided herein are methods for treating cancer comprising administering to a checkpoint inhibitor refractory subject composition comprising a therapeutically effective amount of an expression vector comprising a polynucleotide sequence encoding a chimeric CD154. In certain embodiments, the polynucleotide sequence comprises a first nucleotide sequence encoding an extracellular domain of nonhuman CD154 that replaces a cleavage site of human CD154, and a second nucleotide sequence encoding an extracellular domain of human CD154 that binds to a CD154 receptor.

In certain embodiments, the polynucleotide sequence comprises SEQ ID NO: 1. In certain embodiments, the expression vector comprises viral DNA. In certain embodiments, the viral DNA is selected from the group consisting of adenoviral DNA and retroviral DNA. In certain embodiments, the expression vector comprises a promoter sequence. In certain embodiments, the expression vector comprises a polyadenylation signal. In certain embodiments, the polynucleotide sequence encoding a chimeric CD154 comprises SEQ ID NO: 1. In certain embodiments, the expression vector comprises SEQ ID NO: 1 operatively linked to a promoter sequence and to a polyadenylation signal sequence.

In certain embodiments, the expression vector is present as a pharmaceutical composition.

In certain embodiments, the subject is refractory to treatment with a PD-1 inhibitor. In certain embodiments, the subject is refractory to treatment with a PD-L1 inhibitor. In certain embodiments, the subject is refractory to treatment with a PD-L2 inhibitor.

In certain embodiments, the subject is a PD-L1 low expressor. In certain embodiments, the subject is a PD-L2 low expressor.

In certain embodiments, the subject has a solid tumor cancer. In certain embodiments, the solid tumor cancer is selected from the group consisting of melanoma, non-small cell lung cancer, renal cell carcinoma, castration-resistant prostate cancer, colon cancer, gastric cancer, pancreatic cancer, head and neck cancer, triple negative breast cancer, glioblastoma, hepatocellular carcinoma, bladder cancer, and ovarian cancer. In certain embodiments, the cancer is a metastatic cancer. In certain embodiments, the cancer is metastatic melanoma.

In certain embodiments, the subject has a hematological cancer. In certain embodiments, the hematological cancer is selected from the group consisting of acute myeloid leukemia, chronic lymphocytic leukemia, Hodgkin lymphoma, non-Hodgkin lymphoma, and multiple myeloma.

In certain embodiments, the subject is identified as a checkpoint inhibitor refractory subject. In certain embodiments, the identification comprises (i) providing a test tissue sample obtained from the subject, wherein the test tissue sample comprising cancer cells and/or tumor-infiltrating inflammatory cells, (ii) assessing the proportion of cells in the test tissue sample that express PD-L1 and/or PD-L2 on the cell surface, and (iii) identifying the subject as a checkpoint inhibitor refractory subject based on an assessment that the proportion of cells in the test tissue sample that express PD-L1 and/or PD-L2 on the cell surface exceeds or falls below a predetermined threshold level.

In certain embodiments, the administration results in increased expression of one or more of PDI, PD-L 1, or PD-L2. In certain embodiments, the administration sensitizes the cancer to treatment with a checkpoint inhibitor.

In certain embodiments, provided herein are methods of administering to a checkpoint inhibitor refractory subject an expression vector comprising a polynucleotide sequence encoding a chimeric CD154, and a composition comprising a therapeutically effective amount of at least one checkpoint inhibitor. In certain embodiments, the checkpoint inhibitor is a PD-1 pathway inhibitor. In certain embodiments, the checkpoint inhibitor is selected from the group consisting of PD-1 inhibitors, PD-L1 inhibitors, and PD-L2 inhibitors. In certain embodiments, the PD-1 inhibitor is selected from the group consisting of macrocyclic peptides, pembrolizumab, lambrolizumab, nivolumab, pidilizumab, AMP-224, MEDI0680, and AUNP-12. In certain embodiments, the checkpoint inhibitor is a PD-L1 inhibitor. In certain embodiments, the PD-L1 inhibitor is selected from the group consisting of durvalumab (MEDI4736), atezolizumab (MPDL3280A), MEDI473, MSBOOI0718C, BMS935559 (MDX-II05), and BMS936559. In certain embodiments, the checkpoint inhibitor is a PD-L2 inhibitor.

### DETAILED DESCRIPTION

Unless defined otherwise, all technical and scientific terms used herein have the same meaning as is commonly understood by one of skill in the arts to which the invention belongs. Unless specific definitions are provided, the nomenclature utilized in connection with, and the procedures and techniques of, analytical chemistry, synthetic organic chemistry, and medicinal and pharmaceutical chemistry described herein are those well-known and commonly used in the art. In the event that there is a plurality of definitions for terms herein, those in this section prevail. Standard techniques may be used for chemical synthesis, chemical analysis, pharmaceutical preparation, formulation and delivery, and treatment of subjects. Certain such techniques and procedures may be found for example in “Carbohydrate Modifications in Antisense Research” Edited by Sangvi and Cook, American Chemical Society, Washington D.C., 1994; and “Remington's Pharmaceutical Sciences,” Mack Publishing Co., Easton, Pa., 18th edition, 1990; and which is hereby incorporated by reference for any purpose. Where permitted, all patents, patent applications, published applications and publications, GENBANK sequences, websites and other published materials referred to throughout the entire disclosure herein, unless noted otherwise, are incorporated by reference in their entirety. Where reference is made to a URL or other such identifier or address, it is understood that such identifiers can change and particular information on the internet can change, but equivalent information can be found by searching the internet. Reference thereto evidences the availability and public dissemination of such information.

Before the present compositions and methods are disclosed and described, it is to be understood that the terminology used herein is for the purpose of describing particular embodiments only and is not intended to be limiting. It must be noted that, as used in the specification and the appended claims, the singular forms “a,” “an” and “the” include plural referents unless the context clearly dictates otherwise.

### Definitions

“Checkpoint inhibitor” means any compound capable of inhibiting an immune checkpoint molecule. Inhibition may be partial or complete inhibition. In some embodiments, a checkpoint inhibitor inhibits the interaction between PD-1 and either or both of its ligands, PD-L1 and PD-L2.

“Checkpoint inhibitor refractory subject” means a subject having cancer who does not experience a clinically acceptable response following treatment with a checkpoint inhibitor.

“Subject in need thereof” means a subject that is identified as in need of therapy or treatment.

“Program cell death protein I” or “PD-1” means a protein that in humans is encoded by the PDCD1 gene that is a cell surface receptor that belongs to the immunoglobulin superfamily and is expressed on T cells and pro-B cells. PD-1, functioning as an immune checkpoint, plays an important role in down regulating the immune system by preventing the activation of T-cells, which in turn reduces autoimmunity and promotes self-tolerance. The inhibitory effect of PD-L1 is accomplished through a dual mechanism of promoting apoptosis (programmed cell death) in antigen specific T-cells in lymph nodes while simultaneously reducing apoptosis in regulatory T cells. The complete PD-1 protein sequence can be found under GENBANK® Accession No. NP 005009.

“Program cell death protein I ligand I” or “PD-L1” means variants, isoforms, and species homologs of the transmembrane protein that is a ligand for PD-1. The complete PD-L1 sequence can be found under GENBANK® Accession No. NP_054862.

“Program cell death protein I ligand 2” or “PD-L2” means variants, isoforms and species homologs of a transmembrane protein that is a ligand for PD-1. The complete PD-L2 sequence can be found under GENBANK® Accession No. NP 079515.2.

“PD-L1 low expressor” means a subject whose cancer cells express a level of PD-L1 lower than that required to achieve a clinically acceptable response following treatment with an inhibitor of the PD-1 pathway.

“PD-L2 low expressor” means a subject whose cancer cells express a level of PD-L2 lower than that required to achieve a clinically acceptable response following treatment with an inhibitor of the PD-1 pathway.

“Corresponding” means the sequence of nucleotides or amino acids of gene or protein of one species that is homologous to the nucleotide or amino acid sequence of a gene or protein of another species. The homology may be based on the similarity in secondary structure. For example, homology may be based on the location of domain boundaries among CD154 of different species.

“Chimeric CD154” means a ligand comprised of at least one domain or subdomain ofCD154 from one species and at least one domain or subdomain ofCD154 from a different species. The at least two species from which the chimeric CD154 is derived may be human and murine CD154. In some embodiments, a chimeric CD154 is ISF35.

“Immunostimulatory factor 35” or “ISF35” means a chimeric CD154 encoded by the nucleotide sequence of SEQ ID NO: 1.

“Subdomain” means a sequence of at least two amino acids that is part of a domain ofCD154. A subdomain also encompasses an amino acid sequence from which one or more amino acids have been deleted, including one or more amino acids truncated from an end of the sequence.

“Cleavage site” means a sequence of amino acids that is recognized by proteases, typically matrix metalloproteases that cleave CD154 from the surface of the expressing cell. The cleavage site ofCD154 is typically found at or around the boundaries of domains III and IV ofCD154. In some embodiments, one such cleavage site comprises the region approximately between amino acids 108 and 116 of human CD154.

“Less susceptible to cleavage” means a higher resistance of chimeric CD154 to proteolytic cleavage compared to that of native human CD154, as measured by the amount of soluble CD154 generated by a given number of cells over a period of time. In some embodiments, a chimeric CD154 of the present invention is less susceptible to cleavage when it is cleaved at a rate at least 90% less than that of native CD154.

“Expression vector” means a nucleic acid that expresses, or directs the expression of, a recombinant nucleotide sequence and that is capable of infecting cells and replicating itself therein. Typical expression vectors include plasmids used in recombinant DNA technology and various viruses capable of replicating within bacterial or animal cells. A number of expression vectors have been described in the literature. See, for example Cantwell et al., Blood. In (1996) entitled “Adenovirus Vector Infection of Chronic Lymphocytic Leukemia B Cells;” Woll, P. J. and I. R. Hart, Ann. Oncol., 6 Suppl 1.73 (1995); Smith, K. T., A. J. Shepherd, J. E. Boyd, and G. M. Lees, Gene Ther., 3:190 (1996); Cooper. M. J., Semin Oncol., 23: 172(1996); Shaughnessy E., D. Lu, S. Chalterjee, and, K. K. Wong, Semin Oncol., 23: 159 (1996), Glorioso, J. C., N. A, DeLuca, and D. J. Fink, Annu. Rev. Microbiol., 49:675 (1995); Flotte, T. R. and BJ. Carter, Gene Ther., 2:357 (1995); Randrianarison-Jewtoukoff, V. and M. Perricaudet, Biologicals., 23:145 (1995); Kohn, D. B., Curr. Opin. Pediatr., 7:56(1995); Vile, R. G. and S. J. Russell, Br. Med. Bull., 51: 12 (1995); Russell, S. J., Semin. Cancer Biol., 5:437 (1994); and Ali, M., N. R. Lemoine, and C. J. Ring, Gene Ther., 1:367 (1994).

“Subject suspected of having” means a subject exhibiting one or more clinical indicators of a disease.

“Administering” means providing a pharmaceutical agent or composition to a subject, and includes, but is not limited to, administering by a medical professional and self-administering.

“Parenteral administration” means administration through injection or infusion. Parenteral administration includes, but is not limited to, subcutaneous administration, intravenous administration, and intramuscular administration.

“Subcutaneous administration” means administration just below the skin.

“Intravenous administration” means administration into a vein.

“Administered concomitantly” means to the co-administration of two or more agents to a subject in any manner in which the pharmacological effects of each agent are present in a subject. Concomitant administration does not require that both agents be administered in a single pharmaceutical composition, in the same dosage form, or by the same route of administration. The effects of both agents need not be present at the same time. The effects need only be overlapping for a period of time and need not be coextensive.

“Duration” means the period of time during which an activity or event continues. In certain embodiments, the duration of treatment is the period of time during which doses of pharmaceutical agent or pharmaceutical composition are administered. “Therapy” means a disease treatment method. In certain embodiments, therapy includes, but is not limited to, chemotherapy, radiation therapy, or administration of a pharmaceutical agent.

“Treating” or “treatment” means the application of one or more specific procedures used for the cure or amelioration of a disease. In certain embodiments, the specific procedure is the administration of one or more pharmaceutical agents.

“Amelioration” means a lessening of severity of at least one indicator of a condition or disease. In certain embodiments, amelioration includes a delay or slowing in the progression of one or more indicators of condition or disease. The severity of indicators may be determined by subjective or objective measures which are known to those skilled in the art.

“At risk for developing” means the state in which a subject is predisposed to developing a condition or disease. In certain embodiments, a subject at risk for developing a condition or disease exhibits one or more symptoms of the condition or disease, but does not exhibit a sufficient number of symptoms to be diagnosed with the condition or disease. In certain embodiments, a subject at risk for developing a condition or disease exhibits one or more symptoms of the condition or disease, but to a lesser extent required to be diagnosed with the condition or disease.

“Prevent the onset of” means to prevent the development of a condition or disease in a subject who is at risk for developing the disease or condition. In certain embodiments, a subject at risk for developing the disease or condition receives treatment similar to the treatment received by a subject who already has the disease or condition.

“Delay the onset of” means to delay the development of a condition or disease in a subject who is at risk for developing the disease or condition. In certain embodiments, a subject at risk for developing the disease or condition receives treatment similar to the treatment received by a subject who already has the disease or condition.

“Therapeutic agent” means a pharmaceutical agent used for the cure, amelioration or prevention of a disease.

“Dose” means a specified quantity of a pharmaceutical agent provided in a single administration. In certain embodiments, a dose may be administered in two or more boluses, tablets, or injections. For example, in certain embodiments, where subcutaneous administration is desired, the desired dose requires a volume not easily accommodated by a single injection. In such embodiments, two or more injections may be used to achieve the desired dose. In certain embodiments, a dose may be administered in two or more injections to minimize injection site reaction in an individual. In certain embodiments, a dose is administered as a slow infusion.

“Dosage unit” means a form in which a pharmaceutical agent is provided. In certain embodiments, a dosage unit is a vial containing lyophilized oligonucleotide. In certain embodiments, a dosage unit is a vial containing reconstituted oligonucleotide.

“Therapeutically effective amount” refers to an amount of a pharmaceutical agent that provides a therapeutic benefit to a subject. The therapeutic benefit can be, for example, alleviation or amelioration (complete or partial) of the symptoms of the condition of the subject in need of treatment, any other desired improvement in the patient's symptoms, disease or condition, or prophylaxis or delay in the onset of symptoms of the disease or condition. “Pharmaceutical composition” means a mixture of substances suitable for administering to an individual that includes a pharmaceutical agent. For example, a pharmaceutical composition may comprise a sterile aqueous solution.

“Pharmaceutical agent” means a substance that provides a therapeutic effect when administered to a subject. “Active pharmaceutical ingredient” means the substance in a pharmaceutical composition that provides a desired effect.

“Improved organ function” means a change in organ function toward normal limits. In certain embodiments, organ function is assessed by measuring molecules found in a subject's blood or urine. For example, in certain embodiments, improved liver function is measured by a reduction in blood liver transaminase levels. In certain embodiments, improved kidney function is measured by a reduction in blood urea nitrogen, a reduction in proteinuria, a reduction in albuminuria, etc.

“Acceptable safety profile” means a pattern offside effects that is within clinically acceptable limits.

“Side effect” means a physiological response attributable to a treatment other than desired effects. In certain embodiments, side effects include, without limitation, injection site reactions, liver function test abnormalities, renal function abnormalities, liver toxicity, renal toxicity, central nervous system abnormalities, and myopathies. Such side effects may be detected directly or indirectly. For example, increased aminotransferase levels in serum may indicate liver toxicity or liver function abnormality. For example, increased bilirubin may indicate liver toxicity or liver function abnormality.

“Injection site reaction” means inflammation or abnormal redness of skin at a site of injection in an individual.

“Subject compliance” means adherence to a recommended or prescribed therapy by a subject.

“Comply” means the adherence with a recommended therapy by a subject. “Recommended therapy” means a treatment recommended by a medical professional to treat, ameliorate, delay, or prevent a disease.

“Chemotherapeutic agent” means a pharmaceutical compound useful in the treatment of cancer.

“Administered concomitantly” refers to the co-administration of two or more agents to a subject in any manner in which the pharmacological effects of each agent are present in a subject. Concomitant administration does not require that both agents be administered in a single pharmaceutical composition, in the same dosage form, or by the same route of administration. The effects of both agents need not be present at the same time. The effects need only be overlapping for a period of time and need not be coextensive.

Overview

Immunotherapeutic approaches for the treatment of cancer are designed to elicit or enhance antitumor immune responses. For example, treatment with checkpoint inhibitors blocks the activity of checkpoint molecules that cancer cells use to evade the host immune system. Checkpoint inhibitors include agents that block the interaction between PD-1 and one or both of its ligands PD-L1 and PD-L2. Many patients treated with checkpoint inhibitors are refractory or have suboptimal therapeutic responses. For example, patients that are refractory to checkpoint inhibitors may express low levels of PD-L1 or PDL2 on the cancer cells. Studies have shown that patients with increased PD-L1 or PD-L2 expression have improved responses to checkpoint inhibitors (Current Opinion in Pharmacology 2015, 23:32-38).

Treatment of patients with a therapeutic agent that can enhance cancer cell expression of PD-L1 or PD-L2 may provide an improved response to checkpoint inhibitor.

Accordingly, provided herein are methods for the treatment of cancer comprising administering to a checkpoint inhibitor refractory subject an expression vector, comprising a polynucleotide sequence encoding a chimeric CD154. The administration of the expression vector enhances the expression of PD-L1 and/or PD-L2 on the cancer cells, making the cells susceptible to treatment with inhibitors of the PD-1 pathway. The cancer treatment may occur in vivo and/or ex vivo.

In certain embodiments, the expression vector comprises polynucleotide sequence encoding a chimeric CD154 comprises a first nucleotide sequence encoding an extracellular domain of non-human CD154 that replaces a cleavage site of human CD154, and a second nucleotide sequence encoding an extracellular domain of human CDI54 that binds to a CDI54 receptor. In certain embodiments, the polynucleotide sequence of the chimeric CDI54 is SEQ ID NO: I. The chimeric CDI54 encoded by SEQ ID NO: 1 is also known as immunostimulatory factor 35 or ISF35. Compositions and methods related to ISF35 are described, for example, in International Application Publication No. WO/2003/099340, published Dec. 4, 2003.

In certain embodiments, the expression vector comprises viral DNA. In certain embodiments, the expression vector comprises adenoviral DNA. In certain embodiments, the expression vector comprises retroviral DNA.

In certain embodiments, the expression vector comprises a promoter sequence. In certain embodiments, the promoter sequence is a CMV promoter.

In certain embodiments, the expression vector comprises a polyadenylation signal. In certain embodiments, the polyadenylation signal is a bovine growth hormone polyadenylation sequence.

In certain embodiments, the expression vector comprises a polynucleotide sequence encoding a chimeric CD154 operatively linked to a promoter sequence and to a polyadenylation signal sequence. In certain embodiments, the expression vector is present as a pharmaceutical composition. In certain embodiments, the expression vector is present in sterile water. In certain embodiments, the expression vector is present in sterile, normal saline.

In certain embodiments, a subject selected for treatment according to the methods provided herein is refractory to treatment with an inhibitor of the PD-1 pathway. In certain embodiments, a subject treatment is refractory to treatment with a PD-1 inhibitor. In certain embodiments, a subject is refractory to treatment with a PD-L 1 inhibitor. In certain embodiments, a subject is refractory to treatment with a PD-L2 inhibitor.

In certain embodiments, a subject selected for treatment according to the methods provided herein is a PD-L1 low expressor. In certain embodiments, a subject selected for treatment is a PD-L2 low expressor.

In certain embodiments, a subject selected for treatment according to the methods provided herein has a solid tumor cancer. The solid tumor cancer may be selected from the group consisting of melanoma, non-small cell lung cancer, renal cell carcinoma, castration-resistant prostate cancer, colon cancer, gastric cancer, pancreatic cancer, head and neck cancer, triple negative breast cancer, glioblastoma, bladder cancer, ovarian cancer, and hepatocellular carcinoma. In certain embodiments, a subject selected for treatment according to the methods provided herein has a hematological cancer. The hematological cancer may be selected from the group consisting of acute myeloid leukemia, chronic lymphocytic leukemia, Hodgkin lymphoma, non-Hodgkin lymphoma, and multiple myeloma.

In certain embodiments, the cancer is a metastatic cancer. In certain embodiments, the cancer is metastatic melanoma.

In any of the embodiment provided herein, prior to treatment, a subject is identified as a checkpoint inhibitor refractory subject. In certain embodiments, the methods provided herein comprise determining the status of the expression of PD-L1 in the subject. In certain embodiments, the methods provided herein comprise determining the status of the expression of PD-L2 in the subject. In certain embodiments, the identification comprises (i) providing a test tissue sample obtained from the subject, wherein the test tissue sample comprising cancer cells and/or tumor-infiltrating inflammatory cells, (ii) assessing the proportion of cells in the test tissue sample that express PD-L1 and/or PD-L2 on the cell surface, and (iii) identifying the subject as a checkpoint inhibitor refractory subject based on an assessment that the proportion of cells in the test tissue sample that express PD-L1 and/or PD-L2 on the cell surface exceeds or falls below a predetermined threshold level. In certain embodiments, the threshold level is a level present in a subject who did not experience a clinically acceptable response to treatment with an inhibitor of the PD-1 pathway.

The PD-L1 and/or PD-L2 protein levels can be determined in the cancer cells of subject using, for example, one or more methods including but not limited to fluorescence activated cell sorting (FACS), Western blot, enzyme-linked immunosorbent assay (ELISA), immunoprecipitation, immunohistochemistry, immunofluorescence, radioimmunoassay, dot blotting, immunodetection methods, HPLC, surface plasmon resonance, optical spectroscopy, mass spectrometry, quantitative polymerase chain reaction (qPCR), real-time qPCR (RT-qPCR), multiplex qPCR or RT-qPCR, RNA-seq, microarray analysis, serial analysis of gene expression (SAGE), MassARRAY@ technique, and fluorescence in situ hybridization (FISH).

In any of the embodiments provided herein, the subject receives treatment with an expression vector comprising a polynucleotide sequence encoding a chimeric CD154, and a checkpoint inhibitor. In certain embodiments, the expression vector and checkpoint inhibitor are administered concomitantly.

In certain embodiments, a checkpoint inhibitor is a PD-1 inhibitor. In certain embodiments, a checkpoint inhibitor is a PD-L1 inhibitor. In certain embodiments, the checkpoint inhibitor is a PD-L2 inhibitor. In certain embodiments, a PD-1 inhibitor is selected from the group consisting of Keytruda (pembrolizumab; MK3475M; lambrolizumab), Opdivo (nivolumab; MDXII06; BMS936558), pidilizumab (CT-011), AMP-224, MEDI0680 (AMP-514), and AUNP-12. In certain embodiments, a PD-L1 inhibitor is selected from the group consisting of durvalumab (MEDI4736), atezolizumab (MPDL3280A), MSBQOI0718C, BMS935559 (MDX1105), and BMS936559.

The methods provided herein comprise the administration of polynucleotide sequences to a subject. Methods for introducing polynucleotide sequences into specific cells in vivo are well known and include use of expression vectors and direct injection of such vectors into the subject. In a typical application, an expression vector containing a polynucleotide sequence is introduced into the circulation or at a localized site of the subject to allow the vector to specifically infect the desired cells. In other embodiments the vector is injected directly into a solid tumor that contains at least some of the cells into which the polynucleotide sequence is to be introduced.

In certain embodiments, the methods comprise a first step of first obtaining cancer cells from a subject, inserting therein a polynucleotide sequence described herein, so that a chimeric CD154 is expressed on the surface of the cancer cells, and re-administering the cells back into the subject. One of ordinary skill in the art will understand that numerous methods are applicable for re-administering the transformed cancer cells into the subject.

Also provided herein are methods for predicting the therapeutic effectiveness of an inhibitor of the PD-1 pathway in a subject, which method comprises: (a) optionally providing a test tissue sample obtained from a patient with cancer of the tissue, the test tissue sample comprising tumor cells and tumor-infiltrating inflammatory cells; (b) assaying the test tissue sample to determine the proportion of cells therein that express PD-L1 on the cell surface; (c) comparing the proportion of cells that express PD-L1 on the cell surface with a predetermined threshold value; and (d) predicting the therapeutic effectiveness of the checkpoint inhibitor, wherein if the proportion of cells that express PD-L1 on the cell surface exceeds the threshold proportion the inhibitor is predicted to be effective in treating the patient, and wherein if the proportion of cells that express PD-L1 on the cell surface is below the threshold proportion the inhibitor is predicted to not be effective in treating the patient.

Suitable cancer types include but are not limited to acute lymphoblastic leukemia, acute myeloid leukemia, adrenocortical carcinoma, aids-related cancers, AIDS-related lymphoma, anal cancer, astrocytoma, basal cell carcinoma, bile duct cancer, bladder cancer, bone cancer, brain stem glioma, brain tumor, breast cancer, bronchial adenomas, Burkitt's lymphoma, carcinoid tumor, carcinoma, central nervous system lymphoma, cerebellar astrocytoma, cervical cancer, chronic lymphocytic leukemia, chronic myelogenous leukemia, chronic myeloproliferative disorders, colon cancer, colorectal cancer, cutaneous t-cell lymphoma, endometrial cancer, ependymoma, esophageal cancer, extragonadal germ cell tumor, eye cancer, intraocular metanoma, eye cancer, retinoblastoma, gallbladder cancer, gastrointestinal carcinoid tumor, gastrointestinal stromal tumor (gist), germ cell tumor (extracranial), germ cell tumor (extragonadal), germ cell tumor (ovarian), gestational trophoblastic tumor, glioma; hairy cell leukemia, head and neck cancer, hepatocellular (liver) cancer, Hodgkin's lymphoma, hypopharyngeal cancer, hypothalamic and visual pathway glioma, intraocular melanoma, islet cell carcinoma (endocrine pancreas), Kaposi's sarcoma, kidney (renal cell) cancer, laryngeal cancer, lip and oral cavity cancer, liver cancer, lung cancer (non-small cell), lung cancer (small cell), lymphoma, (cutaneous t-cell), lymphoma (non-Hodgkin's), malignant fibrous histiocytoma of bone/osteosarcoma, medulloblastoma, melanoma, Merkel cell carcinoma, mesothelioma, metastatic squamous neck cancer with occult primary, multiple endocrine neoplasia syndrome, multiple myeloma/plasma, cell neoplasm, mycosis fungoides, myelodysplastic syndromes, myelodysplastic/myeloproliferative diseases, myelogenous leukemia, myeloid leukemia, myeloproliferative disorders, nasal cavity and paranasal sinus cancer, nasopharyngeal cancer, neuroblastoma, oral cancer, oropharyngeal cancer, osteosarcoma/malignant fibrous histiocytoma of bone, ovarian cancer, ovarian epithelial cancer, ovarian germ cell tumor, ovarian low malignant potential tumor, pancreatic cancer, parathyroid cancer, penile cancer, pheochromocytoma, pineoblastoma and supratentorial primitive neuroectodermal tumors, pituitary tumor, plasma cell neoplasm/multiple myeloma, pleuropulmonary blastoma, prostate cancer, rectal cancer, retinoblastoma, rhabdomyosarcoma, salivary gland cancer, sezary syndrome, skin cancer (non-melanoma), melanoma, small intestine cancer, soft tissue sarcoma, squamous cell carcinoma, stomach (gastric) cancer, T-cell lymphoma, testicular cancer, thymoma, thyroid cancer, trophoblastic tumor, gestational, urethral cancer, uterine cancer, endometrial, uterine sarcoma, vaginal cancer, visual pathway and hypothalamic glioma, vulvar cancer, Waldenstrom's macroglobulinemia, and Wilms' tumor.

Certain CD154 Expression Vectors

Provided herein are methods comprising administering certain CD154 expression vectors for the treatment of cancer.

Given the similarities in nucleotide sequences coding for CD154 molecules of different species, such as human, mouse and cow, a nucleotide sequence encoding one domain or subdomain ofCD154 from one species is interchangeable with the corresponding nucleotide sequence ofCD154 from another species to result in a hybrid polynucleotide sequence that encodes a chimeric CD154. One such chimeric molecule is immunostimulatory factor 35 (ISF35), a mouse/human CD154 chimera encoded by the nucleotide sequence of SEQ ID NO: I.

In certain embodiments, the nucleotide sequences that are exchanged for corresponding sequences between species are selected for functional reasons, i.e., because the selected sequence encodes a domain or subdomain that cither provides or modifies a desired function, or eliminates an undesired function of the target ligand gene.

A portion of the human CD154 may cleaved from the parent molecule and become a soluble molecule; this soluble form is generally undesirable. Thus, in some embodiments, exchanging an amino acid, or amino acid sequence, of human CD154 that comprises a cleavage site recognized by proteolytic enzymes with an amino acid, or amino acid sequences of non-human CD154, that does not contain this cleavage site, can at least partially ameliorate the undesirable cleavage. Preferably, the non-human CD154 is murine CD154.

In certain embodiments, an extracellular domain of human CD154 includes at least one amino acid, or a sequence of amino acids, at or near the border of domain III and domain IV that is recognized and cleaved by cleavage proteases. In certain such embodiments, at least one such cleavage site exists between nucleotides 322-348, or amino acids 108-116, of human CD154.

In certain embodiments, an extracellular domain of human CD154 can include at least one amino acid, or a sequence of amino acids, that binds to a human CD154 receptor, e.g., CD40. For this reason, in some embodiments, even the soluble form ofCD154 is capable of binding CD154 receptors on antigen presenting cells and may activity participate in an immune response. Thus, this extracellular region of human CD154 in certain embodiments are preferably conserved in order to maintain native CD 154 receptor binding.

In certain embodiments, a chimeric CD154 polynucleotide sequence comprises a first nucleotide sequence encoding an extracellular subdomain of non-human CD154 that corresponds to and are replaced a cleavage site of human CD154. In some embodiments, replacing a subdomain of human CD154 containing a CD154 cleavage site with the corresponding subdomain of non-human CD154 results in a chimeric CD154 that is less, and in some embodiments substantially less, susceptible to cleavage than human CD154.

In certain embodiments, the first nucleotide sequence may be operatively linked to a second nucleotide sequence that encodes an extracellular sub-domain of human CD154 involved in binding to a human CD154 receptor, such as the CD40 ligand. In this way, the polynucleotide sequence of some embodiments provided by the present invention can encode a chimeric CD154 that binds to human cells expressing the CD154 receptor.

In some embodiments, an extracellular domain of murine and human CD154 can include at least one amino acid, or a sequence of amino acids, that allows expression of the molecule on the membranes of murine and human cells. For example, both murine and human CD154 are expressed by HeLa cells. However, murine CD154 is expressed by a greater variety of cells, including human cells, as compared to human CD154. In fact, murine CD154 may be expressed in human cells that typically do not express human CD154, such as human CD40+ cells, particularly CLL cells. Thus, in some embodiments, exchanging an amino acid, or sequence thereof, of human CD154 that is involved in expression of the human molecule with an amino acid, or sequence thereof, of murine CD154 involved in expression of the non-human molecule can increase the number of cell types which express CD154. Accordingly, in any of the embodiments provided herein, a chimeric CD154 polynucleotide sequence comprises a first nucleotide sequence that further encodes an extracellular subdomain of murine CD154 that is critical for expression of the murine CD154 molecule by murine and human cells. In this way, the polynucleotide sequence provided by some embodiments of the present invention can encode a chimeric CD154 that is capable of expression by a variety of cell types, including human CD40+ cells that do not typically express human CD154. Although this embodiment may involve the use of murine CD154, in some embodiments, other non-human CD154 that may be expressed by human cells can be used.

In certain embodiments, an extracellular domain of murine CD154 can include an amino acid, or a sequence of amino acids, involved in detecting the expression of the chimeric CD154 because it binds to murine CD154 specific antibody. In this way, the expression of the chimeric CD154 polynucleotide sequence can be specifically detected, typically by FACS or immunohistochemistry, and thereby be distinguished from expression of native human CD154.

In certain embodiments, the chimeric CD154 polynucleotide sequence comprises a first nucleotide sequence that further encodes an extracellular subdomain of non-human CD154 that detects the expression of chimeric CD154 by binding to anti-murine CD154 antibodies.

In certain embodiments, this first nucleotide sequence can encode a subdomain of domain IV of non-human CD154, preferably murine, although other sequences may be used alone or in conjunction. This subdomain IV of murine CD154 comprises the amino acid sequences that replace the cleavage site of human CD154, that are thought to be critical for expression of the murine molecule by murine and human cells, and that are involved in detection of the chimeric CD 154 in some embodiments of the present invention. In addition, and in some embodiments, this first nucleotide sequence may encode a subdomain of domain III of non-human CD154 that is at or immediately adjacent to the border of domains III and domain IV. According to some embodiments, this subdomain comprises a portion of cleavage site of human CD154.

In certain embodiments, the first nucleotide sequence can further encode domains I, II and III of murine CD154, because this construct is believed to result in improved expression of the chimeric CD154 by human cells. In some embodiments of the invention, the first nucleotide sequence may encode domain III or a subdomain thereof, of murine CD154; and/or domain II, or subdomain thereof, of murine CD154; and/or domain I, or a subdomain thereof of murine CD154.

Methods of preparing CD154 chimeras, as well as the specific CD154 chimera ISF35 nucleic acid and protein sequences, will be apparent to one of skill in the art, and are described in U.S. Patent Application No. 2003/0220473 to Prussak, and/or U.S. Patent Application No. 2005/0048476, each of which is incorporated herein by reference in its entirety, including for the disclosure of methods of preparing CD154 chimeras.

In certain embodiments, an extracellular domain of ISF35 can include at least one amino acid, or a sequence of amino acids, that may bind to anti-CD 154 antibodies, and thereby neutralize or partially neutralize the immune-activating effect of the ligand. This amino acid or amino acid sequence in some embodiments is preferably the same or substantially similar to the regions in the tertiary structure of CD154 that bind to CD40, CD154's cognate receptor. It is believed that murine CD154 elicits a greater response in terms of anti-CD154 antibody production. As such, it is more sensitive than human CD154 to binding and neutralization by anti-CD154 antibodies, resulting in long-term problems with repeated administration of marine CD154 in humans.

In certain embodiments, an ISF35 polynucleotide sequence comprising a second nucleotide sequence of human CD154 that further encodes an extracellular subdomain to which anti-CD154 antibodies bind is provided. In this way, the polynucleotide sequence encodes protein that is not immunogenic upon administration in humans.

In certain embodiments, provided herein are expression vectors or any other genetic constructs that comprise a polynucleotide sequence of the provided herein capable of expressing ISF35 in a target cell.

In certain embodiments, an expression vector comprises a polynucleotide sequence encoding ISF35 operatively linked to a suitable transcriptional or translation regulatory nucleotide sequence, such as one derived from a mammalian, microbial, viral, or insect gene. Such regulatory sequences include sequences having a regulatory role in gene expression, such as a transcriptional promoter or enhancer, an operator sequence to control transcription, a sequence encoding a ribosomal binding site within the messenger RNA, and appropriate sequences which control transcription, translation initiation, or transcription termination.

Particularly useful regulatory sequences include the promoter regions from various mammalian, viral, microbial, and insect genes. The promoter region directs an initiation of transcription through and including the polynucleotide sequence encoding ISF35. Useful promoter regions include the promoter found in the Rous Sarcoma Virus (RSV) long terminal repeat (LTR), human cytomegalovirus (CMV) enhancer/promoter region, lac promoters, promoters isolated from adenovirus, and any other promoter known by one of ordinary skill in the art would understand to be useful for gene expression in eukaryotes, prokaryotes, viruses, or microbial cells. Other promoters that are particularly useful for expressing genes and proteins within eukaryotic cells/include mammalian cell promoter sequences and enhancer sequences such as those derived from polyoma virus, adenovirus, simian virus 40 (SV40), and the human cytomegalovirus. Particularly useful are the viral early and late promoters, which are typically found adjacent to the viral origin of replication in viruses such as the SV40. One of ordinary skill in the art will understand that the selection of a particular useful promoter depends on the exact cell lines and the other various, parameters of the genetic construct to be used to express a polynucleotide sequence within a particular cell line.

Certain genetic constructs contemplated by the present invention therefore include a polynucleotide sequence operatively linked to either a promoter sequence or a promoter and enhancer sequence and also operatively linked to a polyadenylation sequence that directs the termination and polyadenylation of messenger RNA. In certain embodiments, the expression vector comprising a polynucleotide encoding a chimeric CD154 is constructed using the CMV promoter and the bovine growth hormone polyadenylation sequence.

Certain Dosages

Therapeutic efficacy and the therapeutically effective amount of an expression vector comprising a polynucleotide encoding a chimeric CD154 may be determined by using standard pharmacological procedures. Such procedures may be performed in experimental animals to determine such doses. The dosage regimen of an expression vector comprising a polynucleotide encoding a chimeric CD154 may be selected in accordance with a variety of factors including type, species, age, weight, sex, medical condition of the patient, the severity of the condition, the route of administration, and the particular composition or formulation employed. In certain embodiments, the chimeric CD154 is ISF35.

In certain embodiments, an expression vector is a viral vector. In certain embodiments, a therapeutically effective amount of viral vector comprising a polynucleotide encoding a chimeric CD154, or a recombinant construct thereof comprising a polynucleotide encoding a chimeric CD154, is in the range of from about IXI07 to about IXI012 viral particles. The concentration of the viral preparation can be from about 5×103 to about 5XI012 viral particles per ml. This dosage regimen can be adjusted to provide the optimal therapeutic response. In certain embodiments, the chimeric CDI54 is ISF35.

In certain embodiments, a therapy herein is administered once per week, once per two weeks, once per three weeks, once per four weeks, or once per month. In certain embodiments, a compound provided herein is administered once per two months or once per three months.

Certain Additional Therapies

Treatments for cancer or any of the conditions listed herein may comprise one or more therapies, in addition to administration of an expression vector encoding a chimeric CD154 and a checkpoint inhibitor. Accordingly, provided herein are methods for treating cancer comprising administering an expression vector encoding a chimeric CD154, a checkpoint inhibitor, and one or more additional therapies. The additional therapy may be administered at the same time, less frequently, or more frequently than a compound or pharmaceutical composition of the invention.

In certain embodiments, the one or more additional therapies is a chemotherapeutic agent. A chemotherapeutic agent may be selected from the group consisting of adriamycin, doxorubicin, epirubicin, 5-fluorouracil, cytosine arabinoside (t1Ara-C″), cyclophosphamide, thiotepa, busulfan, cytoxin, taxoids, (e.g., paclitaxel (Taxol, Bristol-Myers Squibb Oncology, Princeton, N.J.) and doxetaxel (Taxotere, Rhone-Poulenc Rorer, Antony, France)), toxotere, methotrexate, cisplatin, melphalan, vinblastine, bleomycin, etoposide, ifosfamide, mitomycin C, mitoxantrone, vincristine, vinorelbine, carboplatin, teniposide, daunomycin, carminomycin, aminopterin, dactinomycin, mitomycins, esperamicins, melphalan, alemtuzimab, tludarabine, and chlorambucil.

In certain embodiments, the one or more additional therapies is an immunotherapeutic agent. In certain embodiments, an immunotherapeutic agent is rituxan. In certain embodiments, an immunotherapeutic agent is an antibody that blocks cytotoxic T-lymphocyte-associated protein 4.

Dosing of the chemotherapeutic agents may depend on the specific composition, the type of cancer to be treated, the condition of the patient, and other factors. In general, the dose can be administered by oral, intravenous, or other administration method as needed. A typical dosage can be from about 0.01 mg/m2, 0.1 mg/m2, 1 mg/m2.5 mg/m2, 10 mg/m2, 100 mg/m2, 100 mg/m2 to about 500 mg/m2 depending on the type of agent, its purity, and other factors mentioned above.

For fludarabine, a typical dose for injection is about 25 mg/m2 administered intravenously over a period of approximately 30 minutes daily for live consecutive days. Each five-day course of treatment should commence about every 28 days. Dosage may be decreased or delayed based on evidence of hematologic or non-hematologic toxicity.

Alemtuzimab therapy can be initiated at a dose of about 3 mg administered as a 2-hour IV infusion daily. When the alemtuzimab 3 mg daily dose is tolerated (e.g., infusion-related toxicities are £ Grade 2), the daily dose can be escalated to about 10 mg and continued until tolerated. When the 10 mg dose is tolerated, the maintenance dose of alemtuzimab 30 mg can be initiated. The maintenance dose of alemtuzimab is typically about 30 mg/day administered three times per week on alternate days (i.e., Monday, Wednesday, and Friday) for up to about 12 weeks. In most patients, escalation to about 30 mg can be accomplished in about 3-7 days. Dose escalation to the recommended maintenance dose of about 30 mg administered three times per week is often required. Single doses of alemtuzimab greater than about 30 mg or cumulative weekly doses of greater that about 90 mg are typically not be administered since higher doses are associated with an increased incidence of pancytopenia.

Administration of the chemotherapeutic agent chlorambucil is typically by use of an oral dose. The usual oral dosage is 0.1 to 0.2 mg/kg body weight daily for 3 to 6 weeks as required. This usually amounts to 4 to 10 mg per day for the average patient. The entire daily dose may be given at one time.

The typical dose of the chemotherapeutic agent rituximab is about 375 mg/m2 by IV infusion once a week for about 4 to about 8 doses. The range can be from about 5 mg/m² to about 500 mg/m².

In certain embodiments, the one or more additional therapies comprises radiation. In certain embodiments, the one or more therapies is a combination of radiation and a chemotherapeutic agent. In certain embodiments, treatment with an expression vector encoding a chimeric CD154, for example, ISF35, may make a cancer more sensitive to radiation treatment and/or combinations of radiation and chemotherapy.

In certain embodiments, the one or more additional therapies is surgery to remove a portion of the cancer.

The invention is described in more detail in the following examples. These examples are provided by way of illustration and are not intended to limit the invention in any way.

### EXAMPLES

### Example 1: Adenovirus Synthesis

The chimeric ISF35 plasmid is digested with the restriction enzymes Nruf and Sma I to release a DNA fragment containing the CMV promoter from pCDNA3, the ISF35 gene, and the polyadenylation signal from pCDNA3. Following gel purification of this fragment by separation of the digested DNA on a 1% agarose gel, the DNA fragment is ligated into the EcoRV site of the adenoviral shuttle vector MCS (SK) pXCX2. This plasmid is a modification of the plasmid pXCX2 such that the pBluescript polylinker sequence has been cloned into the EI region. Following purification of chimeric ISF-MCS (SK) pXCX2 plasmid, 5 ug of this shuttle plasmid is cotransfected with 5 ug of JM 17 plasmid into 293AC2 cells using the calcium phosphate Profection Kit from Promega according to the manufacturer's instructions. Following transfection, the cells are cultured for 5 days to allow for homologous recombination and viral synthesis. Total cells and supernatant are then harvested and freeze-thawed thrice to release cell-associated virus.

Following the initial viral production, a clonal isolate of the virus is obtained by plaque purification. Briefly, the freeze-thawed viral supernatant is cleared of debris by centrifugation at 1000 rpm in a tabletop centrifuge for 5 minutes. 293AC2 cells grown to confluency in 6 well tissue culture plates are then infected with serial dilutions of the viral supernatant for 1-2 hours. Following infection, the media is aspirated and cells are overlayed with DMEM media containing 4% fetal calf serum and 0.65% agarose held at 56° C. Following 4-6 days incubation, isolated plaques are picked into I ml of media and subsequently used for viral amplification.

Large-scale adenovirus preparations are prepared by successively infecting increasing quantities of 293AC2 cells. Purified adenovirus is then purified over cesium chloride step gradients. This method makes use of cesium chloride gradient for concentrating virus particles via a step gradient, with the densities of 1.45 g/cm³ and 1.20 g/cm³, in which 293AC2 expanded virus samples are centrifuged for 2 hours in a SW40 rotor (Beckman, Brea, Calf) at 25,000 rpm at 4° C. The virus band is isolated using a 27 gauge needle and syringe and desalted using a Sephadex G-25 DNA grade column (Pharmacia, Piscataway, N.J.). The virus is desalted against phosphate-buffered saline containing 10% glycerol and stored at −70° C. The final titer of the virus is determined by anion-exchange HPLC.

### Example 2: In Vitro Regulation of PD-L1 and/or PD-L2 Expression on Cancer Cell Lines

Tumor cells are infected with Ad-ISF35. 24-48 hours later, the cells are harvested and stained for expression of PD-L1 and/or PD-L2 with monoclonal antibodies. Surface expression is quantitated by using a method selected from the group consisting of FACS, Western blot, ELISA, immunoprecipitation, immunohistochemistry, immunofluorescence, radioimmunoassay, dot blotting, immunodetection methods, HPLC, surface plasmon resonance, optical spectroscopy, mass spectrometry, HPLC, qPCR, RT-qPCR, multiplex qPCR or RT-qPCR, RNA-seq, microarray analysis, SAGE, MassARRAY technique, FISH, and combinations thereof. Change in expression of Ad-ISF35 infected cells is compared to either non-infected cells or cells infected with a control adenovirus to determine quantitative change in PD-L1 and/or PD-L2 expression. It is anticipated that infection of cancer cells with Ad-ISF35 increases the expression of PD-L1 and/or PD-L2, thereby enhancing the response of the tumor cells to inhibitors of the PD-1 pathway.

### Example 3: In Vivo Treatment with ISF35 and PD-1, PD-L1, or PD-L2 Antibodies

Tumors are directly injected with Ad-ISF35. 24-48 hours later, tumors are biopsied and cells are harvested and stained for expression of PD-L1 or PD-L2 with monoclonal antibodies. Surface expression is quantitated by using a method selected from the group consisting of FACS, Western blot, ELISA, immunoprecipitation, immunohistochemistry, immunofluorescence, radioimmunoassay, dot blotting, immunodetection methods, HPLC, surface plasmon resonance, optical spectroscopy, mass spectrometry, HPLC, qPCR, RT-qPCR, multiplex qPCR or RT-qPCR, RNA-seq, microarray analysis, SAGE, MassARRAY technique, FISH, and combinations thereof. Expression-L1 and/or PD-L2 is compared to tumor biopsies prior to Ad-ISF35 injection. Patients with induced expression-L I and/or PD-L2 are treated with anti-PD-1, anti-PD-L1, or anti-PD-L2 checkpoint inhibitors. It is anticipated that the induction of PD-L1 and/or PD-L2 expression sensitizes the cancer cells to treatment with an inhibitor of the PD-1 pathway, thereby increasing the therapeutic effectiveness of the PD-1 pathway inhibitor.

### Example 4: Treatment of Cancer Patient with Ad-ISF35 and a Checkpoint Inhibitor

An adenoviral expression vector encoding ISF35 operatively linked to a promoter region and a polyadenylation signal region (Ad-ISF35) is administered to subjects having checkpoint refractory cancer. A patient with cancer is identified as described herein. The patient is given a dose of “IXIO” viral particles by intratumoral injection every three weeks. Concomitantly, the patient is treated with anti-PD-1 mAb every three weeks. The patient is monitored twice per month. By use of this method, the cancer progression decreases.

The particular embodiments disclosed above are illustrative only, as the invention may be modified and practiced in different but equivalent manners apparent to those skilled in the art having the benefit of the teachings herein. For example, the process steps set forth above may be performed in a different order. Furthermore, no limitations are intended to the details of construction or design herein shown, other than as described in the claims below. It is, therefore, evident that the particular embodiments disclosed above may be altered or modified and all such variations are considered within the scope and spirit of the invention. Accordingly, the protection sought herein is as set forth in the claims below.

What is claimed is:

1. A method for treating cancer comprising administering to a checkpoint inhibitor refractory subject composition comprising a therapeutically effective amount of an expression vector comprising a polynucleotide sequence encoding a chimeric CD154.

2. The method of claim 1, wherein the polynucleotide sequence encoding a chimeric CD154 comprises a first nucleotide sequence encoding an extracellular domain of non-human CD154 that replaces a cleavage site of human CD154, and a second nucleotide sequence encoding an extracellular domain of human CD154 that binds to a CDI54 receptor.

3. The method of claim 2, wherein the polynucleotide sequence comprises SEQ ID NO: 1.

4. The method of claim 1, wherein the expression vector comprises viral DNA.

5. The method of claim 4, wherein the viral DNA is selected from the group consisting of adenoviral DNA and retroviral DNA.

6. The method of claim 1, wherein the expression vector comprises a promoter sequence.

7. The method of claim 6, wherein the expression vector comprises a polyadenylation signal.

8. The method of claim 1, wherein the expression vector comprises SEQ ID NO: 1 operatively linked to a promoter sequence and to a polyadenylation signal sequence.

9. The method of claim 1, wherein the expression vector is present as a pharmaceutical composition.

10. The method of claim 1, wherein the checkpoint inhibitor refractory subject is refractory to treatment with a PD-1 inhibitor.

11. The method of claim 1, wherein the subject is refractory to treatment with a PD-L1 inhibitor.

12. The method of claim 1, wherein the subject is refractory to treatment with a PD-L2 inhibitor.

13. The method of claim 1, wherein the subject is a PD-L1 low expressor.

14. The method of claim 1, wherein the subject is a PD-L2 low expressor.

15. The method of claim 1, wherein the subject has a solid tumor cancer.

16. The method of claim 15, wherein the solid tumor cancer is selected from the group consisting of melanoma, non-small cell lung cancer, renal cell carcinoma, castration-resistant prostate cancer, colon cancer, gastric cancer, pancreatic cancer, head and neck cancer, triple negative breast cancer, glioblastoma, bladder cancer, ovarian cancer, and hepatocellular carcinoma.

17. The method of claim 1, wherein the subject has a hematological cancer.

18. The method of claim 17, wherein the hematological cancer is selected from the group consisting of acute myeloid leukemia, chronic lymphocytic leukemia, Hodgkin lymphoma, non-Hodgkin lymphoma, and multiple myeloma.

19. The method of claim 1, further comprising, prior to treatment, identifying the subject as a checkpoint inhibitor refractory subject.

20. The method of claim 19, wherein the identifying comprises:
(i) providing a test tissue sample obtained from the subject, wherein the test tissue sample comprising cancer cells and/or tumor-infiltrating inflammatory cells,
(ii) assessing the proportion of cells in the test tissue sample that express PD-L1 and/or PD-L2 on the cell surface, and
(iii) identifying the subject as a checkpoint inhibitor refractory subject based on an assessment that the proportion of cells in the test tissue sample that express PD-L1 and/or PD-L2 on the cell surface exceeds or falls below a predetermined threshold level.

21. The method of claim 1, wherein the administration results in increased expression of one or more of PD-1, PD-L1, or PD-L2.

22. The method of claim 1, wherein the administration sensitizes the cancer to treatment with a checkpoint inhibitor.

23. The method of claim 1, further comprising administering a composition comprising a therapeutically effective amount of at least one checkpoint inhibitor.

24. The method of claim 23, wherein the checkpoint inhibitor is a PD-1 pathway inhibitor.

25. The method of claim 23, wherein the checkpoint inhibitor is selected from the group consisting of PD-L1 inhibitors, PD-L1 inhibitors, and PD-L2 inhibitors.

26. The method of claim 25, wherein the PD-L1 inhibitor is selected from the group consisting of a macrocyclic peptide, pembrolizumab, lambrolizumab, nivolumab, pidilizumab, AMP-224, MEDI0680, and AUNP-12.

27. The method of claim 23, wherein the checkpoint inhibitor is a PD-L1 inhibitor.

28. The method of claim 27, wherein the PD-L1 inhibitor is selected from the group consisting of durvalumab (MEDI4736), atezolizumab (MPDL3280A), MEDI473, MSBOOI0718C, BMS935559 (MDX-I105), and BMS936559.

29. The method of claim 23, wherein the checkpoint inhibitor is a PD-L2 inhibitor.
